(19) United States
(12) Patent Application Publication
(10) Pub. No.: US 2005/0192433 A1
(43) Pub. Date: Sep. 1, 2005

(54) Metallic salt of N4-acylcytidine derivatives, and a method for producing N4-acylcytidine derivatives using the same

(75) Inventors: Hideki Umetani, Mobara-shi (JP); Hiroki Ishibashi, Omuta-shi (JP); Atsushi Sakuma, Omuta-shi (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: MITSUI CHEMICALS, INC., Tokyo (JP)

(21) Appl. No.: 11/056,293

(22) Filed: Feb. 14, 2005

(30) Foreign Application Priority Data
Feb. 26, 2004 (JP) 2004-50669

(51) Int. Cl. C07H 19/067; C07H 19/073

(52) U.S. Cl. 536/28.51

(57) ABSTRACT

The present invention provides a method for producing a high purity N⁴-acylcytidine derivative. More specially, the invention provides a compound represented by the formula (1): 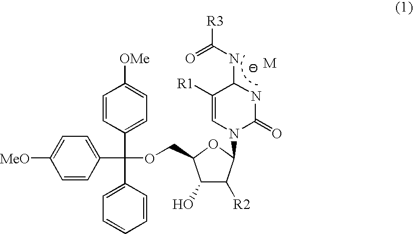
wherein R1 represents a hydrogen atom, an alkyl group having 1 to 4 carbon atoms, an alkenyl group having 2 to 4 carbon atoms, an alkynyl group having 2 to 4 carbon atoms, a perfluoroalkyl group having 1 to 4 carbon atoms, or a halogen atom; R2 is a hydrogen atom, an alkoxyl group having 1 to 4 carbon atoms, an alkoxyl group having 1 to 4 carbon atoms with a substituent, or a halogen atom; R3 represents a methyl group or a phenyl group; and M represents a positive ion of an alkali metal or an alkaline earth metal, and a compound represented by the formula (2): 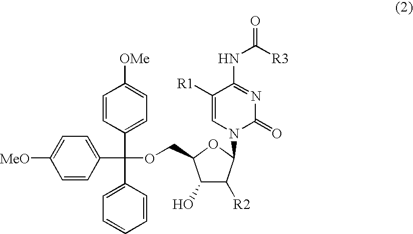 wherein R1, R2, and R3 are as defined above, produced by using the compound represented by the formula (1).

### BACKGROUND OF THE INVENTION

1. Field of the Invention

The present invention relates to a method for producing N⁴-acylcytidine derivatives, which is used as an important intermediate for recently developed antisense DNA drugs.

2. Description of the Related Art

In recent years, the antisense DNA drugs have been developing rapidly accompanying the progress in genomic drug production. As a result, the consumption in raw materials such as DNA oligomers and nucleotide derivatives is growing.

A nucleotide derivative for instance, N⁴-acylcytidine derivative such as N⁴-benzoyl-5′-O-(4,4′-dimethoxytrityl)-2′-deoxy-5-methylcytidine is one of the most important pharmaceutical intermediates for producing an antisense DNA. Moreover, such a pharmaceutical intermediate requires high purity.

Examples of conventional methods for purifying N⁴-acylcytidine derivative such as N⁴-benzoyl-5′-O-(4,4′-dimethoxytrityl)-2′-deoxy-5-methylcytidine are as follows:

(1) The method in which a reaction mixture is washed with sodium bicarbonate, followed by pulverizing the compound using ether/n-hexane (Non-Patent Document 1) and (2) The method in which a reactant is purified using column chromatography (Non-Patent Documents 2 and 3).

However, even after the additional method (1) is conducted to a targeted compound prior to measuring purity in a high-performance liquid chromatography (hereinafter referred to as “HPLC”), the target compound shows 77.0% (in HPLC AREA) purity, which is far from satisfying the condition as a pharmaceutical intermediate. Method (2) is one of the best ways to purify a targeted compound, however purification using column chromatography, as its necessity to large amount of solvent or vacuum evaporation process, is hardly a satisfactory to industrialized form.

In light of these problems, an efficient method to produce high purity N4-acylcytidine derivatives was inevitable.

[Non-Patent Document 1] Nucleic Acids Research, Vol. 15, No. 1, pp. 219-232, 1987)

[Non-Patent Document 2] Photochemistry and Photobiology, Vol. 45, No. 5, pp. 571-574, 1987)

[Non-Patent Document 3] Chemical Pharmaceutical Bulletin, Vol. 34, No. 1, pp. 51-60, 1986)

### SUMMARY OF THE INVENTION

Accordingly, it is an object of the present invention to provide a method to produce a high purity N4-acylcytidine derivative.

After reviewing the results thoroughly to overcome the aforementioned problems, the inventors have concluded that using a metallic salt of 5′-O-(4,4′-dimethoxytrityl)-N⁴-acylcytidine derivative is a possible means to solve the problems. Specifically, it has been found that a reaction mixture containing 5′-O-(4,4′-dimethoxytrityl)-N⁴-acylcytidine derivative can be reacted with a metal hydroxide, or with a metal halide in the presence of an organic amine in order to isolate a metallic salt of the corresponding N⁴-acylcytidine derivative in high purity. The metallic salt of a cytidine derivative is a novel compound, which brings out the uniqueness of the invention. Furthermore, it has been proved that reacting a metallic salt of N⁴-acylcytidine derivative with acid produces a target compound, N⁴-acylcytidine derivative, while maintaining a high purity state, to thus complete the present invention.

That is, the present invention relates to:
1. A compound represented by the formula (1): 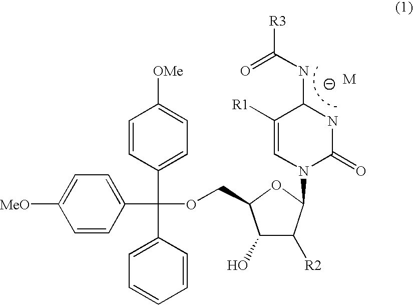
   wherein R1 represents a hydrogen atom, an alkyl group having 1 to 4 carbon atoms, an alkenyl group having 2 to 4 carbon atoms, an alkynyl group having 2 to 4 carbon atoms, a perfluoroalkyl group having 1 to 4 carbon atoms, or a halogen atom; R2 is a hydrogen atom, an alkoxyl group having 1 to 4 carbon atoms, an alkoxyl group having 1 to 4 carbon atoms with a substituent, or a halogen atom; R3 represents a methyl group or a phenyl group; and M represents a positive ion of an alkali metal or an alkaline earth metal,

2. A method for producing a compound represented by the formula (1), which comprises reacting a compound represented by the formula (2): 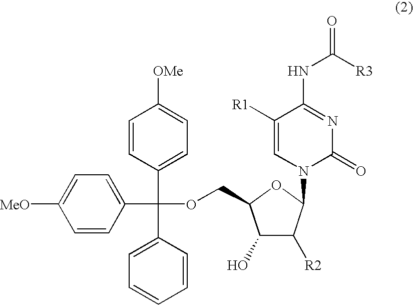
wherein R1, R2, and R3 are as defined above, with a compound represented by the formula (3):
MX (3)
wherein M is as defined in M of the formula (1) and X represents an alkoxide having 1 to 4 carbon atoms, an amide or a negative ion of a hydroxide ion, or with a compound represented by the formula (4):
MY (4)
wherein M is as defined above and Y represents a halide ion, in the presence of an organic amine,

3. A method for producing a compound represented by the formula (2), which comprises reacting a compound represented by the formula (1) with an acid, and

4. A method for producing a compound represented by the formula (2), which comprises reacting a compound represented by the formula (2) with a compound represented by the formula (3), or with a compound represented by the formula (4) in the presence of an organic amine followed by isolating a compound represented by the formula (1), and subsequently reacting the compound represented by the formula (1) with an acid to convert into the compound represented by the formula (2).

According to the present invention, a high purity N⁴-acylcytidine derivative can be easily produced by using metal salts of the corresponding cytidine derivative.

### DESCRIPTION OF THE PREFERRED EMBODIMENTS

The invention is described in detail below.

In the compounds represented by the formula (1) and the formula (2), R1 represents hydrogen atom, an alkyl group having 1 to 4 carbon atoms, an alkenyl group having 2 to 4 carbon atoms, an alkynyl group having 2 to 4 carbon atoms, a perfluoroalkyl group having 1 to 4 carbon atoms, or a halogen atom; R2 represents a hydrogen atom, an alkoxyl group having 1 to 4 carbon atoms, an alkoxyl group having 1 to 4 carbon atoms with a substituent, or a halogen atom; and R3 represents a methyl group or a phenyl group.

Of R2 in the formula (1) and the formula (2), an alkoxyl group having 1 to 4 carbon atoms with a substituent represents an alkoxyl group having the number of carbon atoms in the main chain in the range as above, and having a plurality of substituent in optional positions. Examples of the substituent include an alkoxyl group and an aryl group. Specific examples of the alkoxyl group having 1 to 4 carbon atoms with a substituent include methoxymethoxyl group, butoxymethoxyl group, pentyloxymethoxyl group, trichloroethoxymethoxyl group, methoxyethoxymethoxyl group, methoxyethoxyl group, benzyloxyl group, benzyloxymethyl group, and methoxybenzyloxymethoxyl group.

R2 in the compounds represented by the formula (1) and the formula (2) are not restricted by the isomers, that is both R isomer and S isomer is formable.

The compound represented by the formula (1), depending on a solvent, is produced in forms of solvate or hydrates, which are both acceptable.

Among the compounds represented by the formula (1) used in the invention, preferred is a compound of the formula (1) wherein R1 represents a hydrogen atom or a methyl group, R2 represents a hydrogen atom, and R3 represents a phenyl group, and particularly preferred is a compound of the formula (1) wherein R1 represents a hydrogen atom or a methyl group, R2 represents a hydrogen atom, R3 represents a phenyl group, and M represents a lithium ion. Moreover, among the compounds represented by the formula (2), preferred is a compound of the formula (2) wherein R1 represents a hydrogen atom or a methyl group, R2 represents a hydrogen atom, and R3 represents a phenyl group.

Although a method for obtaining a compound represented by the formula (2), which is a precursor of a compound represented by the formula (1), is not restricted, the compound represented by the formula (2) can be produced via referring to methods illustrated in the aforementioned Non-Patent Document 1, Non-Patent Document 2, and Non-Patent Document 3, including a method which comprises reacting a compound represented by the formula (5): 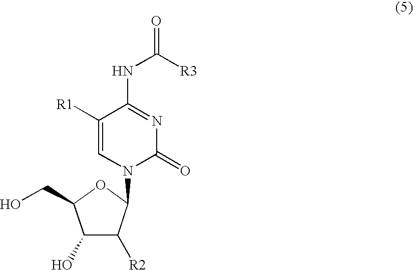
wherein R1, R2, and R3 are as defined above, with 4,4′-dimethoxytritylchloride; and a method which comprises reacting a compound represented by the formula (6): 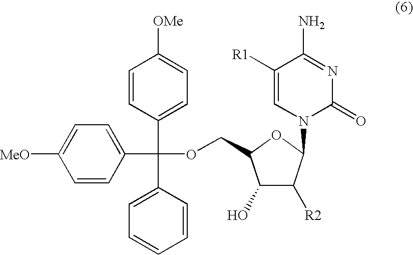
wherein R1 and R2 are as defined above, with a compound represented by the formula (7): 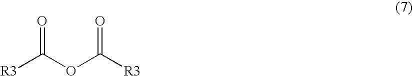
wherein R3 is as defined above, or with a compound represented by the formula (8): 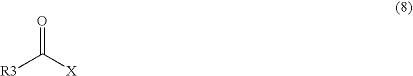
wherein X represents a halogen atom.

The present invention can be used as a purification of the crude product as the compound represented by the formula (2) synthesized by referring to the methods mentioned above.

The crude product of a compound represented by the formula (2) is used as a form which a solvent is distilled off, or a pulverized form, which forms thereof are not particularly restricted. It is possible to use the solvent which was used to produce the compound represented by the formula (2), as dissolved, for the subsequent procedure to produce a compound represented by the formula (1).

A compound represented by the formula (1) can be produced by reacting a compound represented by the formula (2) with a compound represented by the formula (3), or with a compound represented by the formula (4) in the presence of an organic amine.

M in the formula (3) and the formula (4) is a positive ion of an alkali metal or an alkaline earth metal. Among these positive ions, a lithium ion is preferable as the positive ion.

X in the formula (3) is an alkoxide having 1 to 4 carbon atoms, an amide, or a negative ion of a hydroxide ion.

Examples of the amide of X in the formula (3) include diisopropylamide and bis(trimethylsilyl)amide.

X of a compound represented by the formula (3) is preferably isopropoxide, t-butoxide, or a hydroxide ion.

The compound represented by the formula (3) is preferably used in an amount of from 1 to 10 equivalents for economical reasons, although not particularly restricted as long as the compound is more than 1 equivalent.

Y in the formula (4) is a halide ion.

When using the compound represented by the formula (4), it is essential to conduct a procedure in the presence of an organic amine.

The compound represented by the formula (4) is preferably used in an amount of from 1 to 10 equivalents for economical reasons, although not particularly restricted as long as the compound is more than 1 equivalent.

Preferably, although not specified, a secondary amine or a tertiary amine can be used as an organic amine, for example, pyridine, collidine, diisopropylamine, triethylamine, tripropylamine, tributylamine, triamylamine, trihexylamine, triheptylamine, trioctylamine, etc. Particular preference is given to tributylamine, triamylamine, trihexylamine, triheptylamine, and trioctyl amine.

The organic amine is preferably used in an amount of from 1 to 10 equivalents for economical reasons, although not particularly restricted as long as it is more than 1 equivalent.

Reaction solvent used to convert from the compound represented by the formula (2) into the compound represented by the formula (1) is not particularly restricted as long as the compound represented by the formula (1) crystallizes. Examples of the solvent include a ketone solvent such as an acetone, methyl ethyl ketone, and methyl isobutyl ketone; an aromatic solvent such as benzene, toluene, xylene, cumene, cymene, and anisole; an alcohol solvent such as isopropyl alcohol and butanol; a halogen solvent such as methylene chloride, chloroform, and dichloroethane; and a nitrile solvent such as acetonitrile.

Such a solvent is not particularly restricted, and can be used alone or in a mixture of two or more in an optional ratio.

The solvent is generally 3 to 30 times the weight of a substrate, although not particularly restricted as long as it does not affect the reaction.

Reaction temperature, although not restricted as long as the compound represented by the formula (1) is produced and not decomposed, is generally from −10° C. to a boiling point of the solvent used.

Reaction pressure, but not particularly restricted, is generally atmospheric pressure.

In production of a compound represented by the formula (1), a high purity compound represented by the formula (1) can be effectively collected by using the crude compound of a compound represented by the formula (2) and crystallizing the compound represented by the formula (1) to be produced from the reaction solution.

The compound represented by the formula (2) can be produced by reacting the compound represented by the formula (1) with an acid.

Without restriction, as long as degradation of the compound represented by the formula (2) does not occur, an organic acid or inorganic acid can be used as the acid.

It is preferred to use an acetic acid for an organic acid and an hydrochloric acid for an inorganic acid.

Acid usage amount is not restricted, however it is preferred to use pH ranged in 3 and 7 of a reaction solvent to prevent a decomposition of the target compound.

Reaction solvent used in the reaction between the compound represented by the formula (1) and acid has no restriction as long as the solvent does not decompose the target compound to be produced. Examples of the solvent include water; a ketone solvent such as an acetone, methyl ethyl ketone, and methyl isobutyl ketone; an aromatic solvent such as benzene, toluene, xylene, cumene, cymene, and anisole; an alcohol solvent such as methanol, ethanol, isopropyl alcohol and butanol; a halogen solvent such as methylene chloride, chloroform, and dichloroethane; an ester solvent such as ethyl acetate, propyl acetate, isoproryl acetate, and butyl acetate; an ether solvent such as diethyl ether, diisopropyl ether, t-butylmethyl ether, tetrahydrofuran, and dioxane; and a nitrile solvent such as acetonitrile.

Such solvent, without restriction, can be used alone or in a mixture of two or more solvents. In case of using two or more solvents, these solvents may be in homogeneous or in two layers. However, having two layer, if the compound represented by the formula (2) does not crystallize after acid is added to the solution dropwise, it is preferred that the organic layer, containing the compound represented by the formula (2), is separated to be used in the pulverizing process as described below.

Solvent usage amount is not particularly restricted, however it is preferred to use less than 50 times the weight of a substrate. Reaction temperature, although not restricted as long as the compound represented by the formula (2) does not decompose, is preferred to be in the range of −10° to 50° C.

The compound represented by the formula (2), which is obtained by reacting the compound represented by the formula (1) and acid, can be isolated using the method described below.

When the compound represented by the formula (2) is precipitated from the reaction mixture, the target compound can be isolated by filtration.

When the compound represented by the formula (2) is not precipitated from the reaction mixture, it is possible to add a solvent different from that of a reaction mixture in which the compound represented by the formula (2) is dissolved to pulverize the compound, precipitate powders of the compound represented by the formula (2) from the mixture and isolate it by filtration.

When precipitating powders of the compound represented by the formula (2), the reaction mixture containing represented by the formula (2) can be washed in prior with water, an aqueous alkaline solution or brine. Furthermore, the reaction mixture can be used by distilling the solvent off under reduced pressure to concentrate it in an arbitrary concentration.

The solvent used to precipitate powders of the compound represented by the formula (2) is not particularly restricted as long as the reaction solvents are miscible with each other. Such solvents, for example, are water; an alcohol solvent such as methanol, ethanol, and isopropyl alcohol; an aliphatic hydrocarbon solvent such as heptane, hexane, and cyclohexane; and an ether solvent such as diethyl ether, diisopropyl ether, and t-butylmethyl ether.

Such solvent can be used either individually or in two or more combinations.

Preferable combination of the solvent in the reaction mixture and a solvent mixed thereto are listed below. For example, in case the solvent in the reaction mixture is acetonitrile, adding the reaction mixture dropwise to water, alcohol, or alcohol containing water is preferred; and in case the solvent in the reaction mixture is methyl isobutyl ketone, adding the reaction mixture dropwise to hexane or cyclohexane is preferred.

The amount of reaction mixture and the solvent added thereto used in the reaction, although not restricted as long as the product is precipitated, is preferably in the range of 5 times to 120 times the weight of a substrate.

Preferred pulverization temperature is in between −30° C. and the boiling point of a solvent, although not restricted as long as the product does not decompose.

In a case that the compound represented by the formula (2) is not precipitated from the reaction mixture, other conventional method, as follows, of which the solvent containing the compound represented by the formula (2) is washed with water, an aqueous alkaline solution, or brine, followed by drying with a spray drier, etc., is used for pulverization.

Pulverization pressure, but not restricted, is generally atmospheric pressure.

As described above, in the method for producing the compound represented by the formula (2), wherein the crude compound of the compound represented by the formula (2), which is produced from the compound represented by the formula (5) or the formula (6), is reacted with the compound represented by the formula (3), or with the formula (4) in the presence of an organic amine, where a high purity compound represented by the formula (1) is isolated followed by reacting with an acid to convert into the compound represented by the formula (2), the compound represented by the formula (1) is available.

### EXAMPLES

The present invention is hereinafter described in more detail by means of the following Examples, but these examples are not intended to limit the present invention.

In addition, following compounds are written in abbreviation in the specification below: 5′-O-(4,4′-dimethoxytrityl)-2′-deoxy-5-methylcytidine is abbreviated to “(I)”, N⁴-benzoyl-5′-O-(4,4′-dimethoxytrityl)-2′-deoxy-5-methylcytidine is abbreviated to “(II)”, a lithium salt of N⁴-benzoyl-5′-O-(4,4′-dimethoxytrityl)-2′-deoxy-5-methylcytidine is abbreviated to “(III)”; methyl isobutyl ketone is referred to as “MIBK”; and isopropyl alcohol is abbreviated to “IPA”.

HPLC analysis of compounds (II) and (III) are conducted in the conditions, wherein Develosil ODS-MG-5 column (manufactured by Nomura Chemical Co., Ltd. size 4.6×250 mm) is used; the column oven temperature is 40° C.; the eluant contains acetonitrile and an aqueous 100 mM triethylamine acetate solution of 90 to 10; flow rate is 11.0 ml/min.; the observation wave is λ=254 nm.

HPLC analysis of 5′-O-(4,4′-demthoxytrityl)-N⁴-benzoyl-2′-deoxycytidine lithium salt is conducted in the conditions, wherein YMC-Pack CN A-512 column (manufactured by YMC Co., Ltd. size 6.0×250 mm) is used; the column oven temperature is 35° C.; flow rate is 1.0 ml/min.; the observation wave is λ=235 nm. The eluant was used according to the condition of gradient as described below.

Eluant A: 1.15 g of NH₄H₂PO₄ and 0.92 g of (NH₄)₂HPO₄ is dissolved in 2 L of water.

Eluant B: the ratio of 1.5 L of acetonitrile to 0.1 L of methanol to 0.4 L of eluant A is mixed.

Gradient conditions: at 0 minute, 15% of eluant B; at 25 minute, 50% of eluant B; at 60 minute, 85% of eluant B; at 80 minute, 85% of eluant B; at 82 minute, 15% of eluant B; ending the session at 102 minute, 15% of eluant B.

### COMPARATIVE EXAMPLE

### Further Experiment Done According to the Non-Patent Document 1

Azeotropic dehydration was twice done on 0.3 g of N⁴-benzoyl-2′-deoxy-5-methylcytidine using 20 mL of dehydrated pyridine before dissolving in 30 mL of dehydrated pyridine. After adding 328 mg of 4,4′-dimethoxytrylchloride to the solution, the mixture was reacted at room temperature for 10 hours. Methanol was added to the reaction mixture, and stirred for 1 hour, followed by vacuum evaporation. The concentrated residue was diluted with 20 mL of chloroform and 30 mL of 5 wt. % sodium bicarbonate solution. The organic layer was then filtered via membrane filter followed by vacuum evaporation. To the concentrated residue was added 7 mL of diethyl ether, in which the extracted compound was filtered via membrane filter, and added to 100 mL of hexane dropwise, and was stirred for 2 hours. The crystallized compound was filtered, washed with hexane, and dried under vacuumed condition to yield 0.42 g of solid product. The product was analyzed by HPLC to find only 77% (HPLC AREA) of (II).

### Example 1

To 100 mL of MIBK was added 10.6 g of (I), 4.45 g of dicyclohexylamine, and 5.28 g of benzoic anhydride, and was reacted at 70° C. for 4 hours. The precipitated dicyclohexylamine salt of benzoic acid was removed, and filtered solution was cooled to 3° C. To the cooled solution was added 50 mL of 80% ethanol containing dissolved 2.34 g of sodium hydroxide maintaining under 5° C., and was stirred in an ice-cool for 4 hours. The reaction mixture was neutralized with 29 mL of 2 N hydrochloric acid solution, and the aqueous layer was removed followed by vacuum evaporation. To the residue was added 100 mL of MIBK and 100 mL of 5 wt. % sodium carbonate for extraction. The organic layer was then washed with an aqueous saturated ammonium chloride solution, and dried over sodium sulfate. To the reactant, after filtering the sodium sulfate, was added 20 mL of IPA and 0.92 g of lithium hydroxide monohydrate, and stirred overnight at room temperature. Crystallized compound was filtered and dried under reduced pressure. Obtained compound was a solvated product by one molecule of MIBK and one molecule of water to (III) (hereinafter referred to as 1 MIBK.monohydrate of (III)). The product yield was 10.8 g. HPLC analysis showed 99.8% (HPLC AREA) purity, which was a substantial purity of the product.

Subsequently, to 100 mL of acetonitrile was added 50 mL of water, 2.3 mL of acetic acid, and 10.5 g of 1 MIBK.monohydrate of (III) obtained above. The mixture was stirred for 1 hour at room temperature, and the organic layer was extracted. The organic layer was washed with 100 mL of an aqueous saturated sodium bicarbonate solution followed by saturated brine. To 200 mL of 50% methanol solution in the ice-cool was added the above organic layer dropwise slowly. The reactant was stirred for 4 hours under ice-cooling, then the crystallized compound was filtered and dried in vacuo. The product was (II), and had yield of 8.94 g. HPLC analysis showed 99.8% (HPLC AREA) purity, which was substantially high.

In addition, physical properties of 1 MIBK.monohydrate of (III) are as follows.

¹H NMR (DMSO-d₆) (Internal standard: Tetramethylsilane) 0.85 ppm (6H, d, J=6.60 Hz: MIBK), 1.68(3H, s), 2.00(1H, m: MIBK), 2.06(3H, s: MIBK), 2.16(2H, m), 2.29(2H, d, J=6.93 Hz:MIBK), 3.22(2H, m), 3.74(6H, s), 3.89(1H, d, J=3.30 Hz), 4.30(1H, brs), 5.29(1H, s), 6.32(1H, t, J=6.76 Hz), 6.91(4H, d, J=8.57 Hz), 7.2-7.4(12H, m), 7.53(1H, s), 8.12(2H, d, J=6.27 Hz).

Melting Point 191.6-195.4° C. (decomposition)

Water content (Karl Fischer) about 2.0 wt. %

### Example 2

To 100 mL of MIBK was added 10.0 g of (I), 5.0 g of benzoic anhydride, and 100 g of an aqueous 5 wt. % sodium bicarbonate solution, and was reacted at 70° C. for 4 hours. The reactant was cooled to room temperature, and the organic layer was separated. The organic layer was ice-cooled, and was added 50 mL of an aqueous 80% ethanol solution containing 2.21 g of sodium hydroxide dropwise, and was reacted for 4 hours at the same temperature. The mixture was then neutralized near to pH 7 with 25 wt. % acetic acid, and the aqueous layer was removed. To the vacuum evaporated organic layer was added 100 mL of acetonitrile, 40 mL of IPA, 2.26 g of lithium chloride, and was stirred until the mixture was homogeneous. To the mixture was added 6.4 mL tributylamine dropwise, and was stirred at 50° C. for 3 hours. After the reactant was cooled to room temperature, the crystallized compound was filtered and dried under reduced pressure. The product was (III), and had yield of 10.6 g. HPLC analysis showed 99.9% (HPLC AREA) purity. Unlike Example 1, the product did not form solvates.

To 60 mL of acetonitrile containing 30 mL of water, 0.77 mL of acetic acid was added 6.0 g of (III) obtained above. The mixture was stirred for 1 hour at room temperature, and the organic layer was separated. After washing the organic layer with 15 mL of an aqueous saturated sodium bicarbonate solution followed by 30 mL of saturated brine, the layer was slowly added to 120 mL of the ice-cooled aqueous 50% methanol solution dropwise. The reactant was stirred for 4 hours under ice-cooling, then the precipitate was filtered and dried in vacuo. The product was (II), and had yield of 5.7 g. HPLC analysis showed 99.8% (HPLC AREA) purity, which was remarkably high.

In addition, physical properties of the compound (III) are as follows.

¹H NMR (DMSO-d₆)(Internal standard: Tetramethylsilane) 1.69(3H, s), 2.16(2H, m), 3.22(2H, m), 3.74(6H, s), 3.90(1H, m), 4.31(1H, brs), 5.29(1H, s), 6.32(1H, t, J=6.59 Hz), 6.90(4H, d, J=8.24 Hz), 7.2-7.4(12H, m), 7.54(1H, s), 8.14(2H, d, J=6.27 Hz).

Melting Point 210.2-212.2° C. (decomposition)

### Example 3

To 5 mL of MIBK was added 0.43 g of (I), 215 mg of benzoic anhydride, and 5 g of an aqueous 5 wt. % sodium bicarbonate solution, and was reacted at 70° C. for 4 hours. The reactant was cooled to room temperature, and the organic layer was separated. The organic layer was ice-cooled, and was added 3 mL of an aqueous 80% ethanol solution containing 0.1 g of sodium hydroxide dropwise, and was reacted for 4 hours at the same temperature. The mixture was then neutralized with an aqueous 25 wt. % acetic acid solution, and the organic layer was separated. To the vacuum evaporated organic layer was added 5 mL of toluene, 2 mL of IPA, 98 mg of lithium chloride, and was stirred until the mixture was homogeneous. To the mixture was added 276 μl tributylamine dropwise, and was stirred at room temperature over night. The crystallized compound was filtered, dried in vacuo. The obtained product was (III), and HPLC analysis showed 99.8% (HPLC AREA) purity. The solvates did not form within the product, and had yield of 0.24 g. ¹H NMR analysis was consistent with the synthesized product from Example 2.

### Example 4

The experiment was conducted in the same condition as Example 3, except the toluene was replaced to 5 mL of methyl ethyl ketone. HPLC analysis showed 99.9% (HPLC AREA) purity. The obtained product was (III), in which the solvates did not form, and had yield of 0.31 g. ¹H NMR analysis was consistent with the synthesized product from Example 2.

### Example 5

The experiment was conducted in the same condition as Example 3, except the toluene was replaced to 5 mL of acetone. The product was (III), and HPLC analysis showed 99.9% (HPLC AREA) purity. The solvates did not form within the product, and had yield of 0.38 g. ¹H NMR analysis was consistent with the synthesized product from Example 2.

### Example 6

To 5 mL of MIBK was added 0.43 g of (I), 215 mg of benzoic anhydride, and 5 g of an aqueous 5 wt. % sodium bicarbonate solution, and was reacted at 70° C. for 4 hours. The reactant was cooled to room temperature, and the organic layer was separated. The organic layer was ice-cooled, and was added 3 mL of an aqueous 80% ethanol solution containing 0.1 g of sodium hydroxide dropwise, and was reacted for 4 hours at the same temperature. The mixture was then neutralized with an aqueous 25 wt. % acetic acid, and the organic layer was separated. To the vacuum evaporated organic layer was added 10 mL of acetonitrile and 4 mL of IPA, and was stirred. To the mixture was added 61 mg of lithium isopropoxide, and was stirred at an elevated temperature of 50° C. for 2 hours. The reactant was cooled to room temperature, and the crystallized compound was obtained by filtration. The product was (III), and HPLC analysis showed 99.9% (HPLC AREA) purity. The solvates did not form within the product, and had yield of 0.45 g. ¹H NMR analysis was consistent with the synthesized product from Example 2.

### Example 7

0.57 g of 1 MIBK.monohydrate of (III), which was obtained via Example 1, was added to an aqueous 80% methanol solution in the ice-cool followed by neutralization with acetic acid until pH reached 6. The mixture was stirred for 3 hours in the ice-cool, and the crystallized compound was obtained by filtration. The product was (II), and had yield of 0.43 g. HPLC analysis showed 99.8% (HPLC AREA) purity.

### Example 8

To 10 g of 2′-deoxy-5-methylcytidine was added 90 g of DMF and 10.3 g of benzoic anhydride, and was reacted at 40° C. for 5 hours. Next, to the reactant was added 10.9 g of pyridine, which was then cooled to 10° C., and was added 16.7 g of 4,4′-dimethoxytritylchloride, and was reacted for 15 hours at the same temperature. After the reaction ended, 110.4 g of MIBK and 118 g of an aqueous 5 wt. % sodium bicarbonate solution was added to the reactant, and was stirred to extract the organic layer. To the obtained organic layer, which was washed twice with 110 g of water, was added 34 g of ethanol and 15.8 g of an aqueous 30 wt. % sodium hydroxide solution, and was stirred for 1 hour under ice-cooling. The reactant was extracted using 34 g of water followed by washing the reactant with, in the order of, 34 g of water two times, an aqueous 3 wt. % tartaric acid solution, and 17 g of water. To the obtained organic layer, after concentration under vacuum evaporation, was added 202 g of acetonitrile and 80.8 g of IPA and stirred. To the mixture was added 4.5 g of lithium chloride, 9.5 g of tributylamine, and seed crystal, and was stirred at 50° C. for 5 hours followed by 2 hours under ice-cooling to obtain the precipitate. The obtained precipitate was filtered, washed, and dried to obtain 18.5 g (61%) yield of the product (III). Additionally, HPLC analysis showed 99.8% (HPLC AREA) purity.

### Example 9

10 g of N⁴-benzoyl-2′-deoxycytidine was dissolved in 70 g of pyridine, and was cooled to 10° C. and reacted for 5 hours with addition of 11.8 g of 4,4′-dimethoxytritylchloride. Then, 3.2 g of sodium bicarbonate was added and stirred for 1 hour at room temperature. To the reactant, after concentration under vacuum evaporation, was added 120 g of MIBK and 60 g of water, where the organic layer was extracted and washed twice with 3 wt. % brine. To the obtained organic layer, after vacuum evaporation, was added 100 g of acetonitrile and 40 g of isopropyl alcohol and stirred, and was added additional 4 g of lithium chloride, 8.4 g of tributylamine, and seed crystal, and was stirred for 5 hours in the ice-cool. The precipitate was filtered, washed, and dried to obtain 11.6 g (60% yield) of a lithium salt of 5′-O-(4,4′-dimethoxytrityl)-N⁴-benzoyl-2′-deoxycytidine. Furthermore, HPLC analysis showed 99.2% (HPLC AREA) purity.

In addition, identification data for a lithium salt of 5′-O-(4,4′-dimethoxytrityl)-N⁴-benzoyl-2′-deoxycytidine are as follows.

¹H NMR (DMSO-d₆) (Internal standard: Tetramethylsilane) 8.11(dd, 2H), 7.73(d, 1H), 7.42-7.22(m, 13H), 6.91(d, 4H), 6.26(t, 1H), 5.96(m, 1H), 5.36(m, 1H), 4.29(m, 1H), 3.92(1H, m), 3.74(s, 6H), 3.26-3.20(m, 2H), 2.25-2.22(m, 1H), 2.13-2.07(m, 1H).

Therefore, the present invention provides means of producing high purity N⁴-acylcytidine derivative, which is a raw material of antisense DNA drugs.

1. A compound represented by the formula (1): 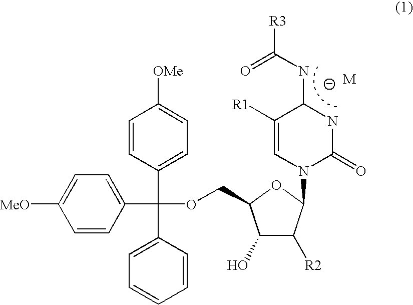
wherein R1 represents a hydrogen atom, an alkyl group having 1 to 4 carbon atoms, an alkenyl group having 2 to 4 carbon atoms, an alkynyl group having 2 to 4 carbon atoms, a perfluoroalkyl group having 1 to 4 carbon atoms, or a halogen atom; R2 is a hydrogen atom, an alkoxyl group having 1 to 4 carbon atoms, an alkoxyl group having 1 to 4 carbon atoms with a substituent, or a halogen atom; R3 represents a methyl group or a phenyl group; and M represents a positive ion of an alkali metal or an alkaline earth metal.

2. The compound according to claim 1, wherein, in the formula (1), R1 represents a hydrogen atom or a methyl group, R2 represents a hydrogen atom, and R3 represents a phenyl group.

3. The compound according to claim 2, wherein, in the formula (1), M represents a lithium ion.

4. A method for producing a compound represented by the formula (1): 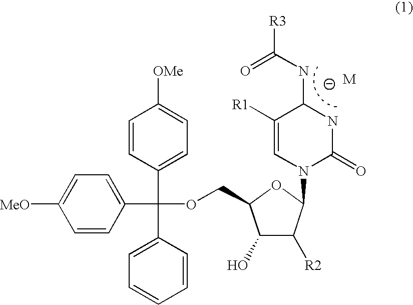
wherein R1 represents a hydrogen atom, an alkyl group having 1 to 4 carbon atoms, an alkenyl group having 2 to 4 carbon atoms, an alkynyl group having 2 to 4 carbon atoms, a perfluoroalkyl group having 1 to 4 carbon atoms, or a halogen atom; R2 is a hydrogen atom, an alkoxyl group having 1 to 4 carbon atoms, an alkoxyl group having 1 to 4 carbon atoms with a substituent, or a halogen atom; R3 represents a methyl group or a phenyl group; and M represents a positive ion of an alkali metal or an alkaline earth metal,
which comprises reacting a compound represented by the formula (2): 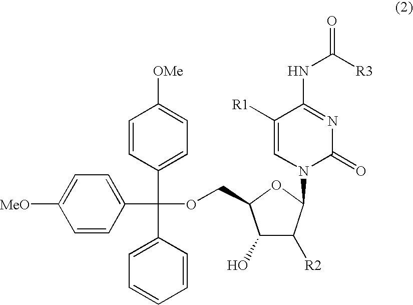
wherein R1, R2, and R3 are as defined in R1, R2, and R3 of the formula (1), with a compound represented by the formula (3):
MX (3)
wherein M is as defined in M of the formula (1) and X represents an alkoxide having 1 to 4 carbon atoms, an amide or a negative ion of a hydroxide ion, or with a compound represented by the formula (4):
MY (4)
wherein M is as defined in M of the formula (1) and Y represents a halide ion, in the presence of an organic amine.

5. A method for producing a compound represented by the formula (2): 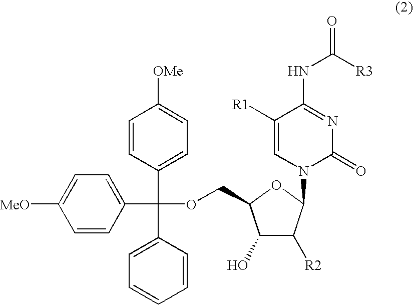
wherein R1 represents a hydrogen atom, an alkyl group having 1 to 4 carbon atoms, an alkenyl group having 2 to 4 carbon atoms, an alkynyl group having 2 to 4 carbon atoms, a perfluoroalkyl group having 1 to 4 carbon atoms, or a halogen atom; R2 is a hydrogen atom, an alkoxyl group having 1 to 4 carbon atoms, an alkoxyl group having 1 to 4 carbon atoms with a substituent, or a halogen atom; and R3 represents a methyl group or a phenyl group,
which comprises reacting a compound represented by the formula (1): 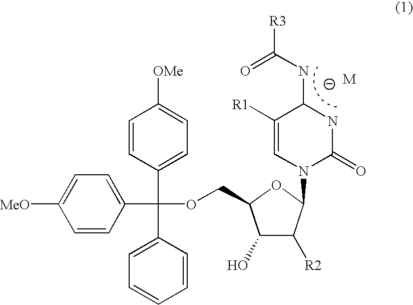
wherein R1, R2, and R3 are as defined in R1, R2, and R3 of the formula (2) and M represents a positive ion of an alkali metal or an alkaline earth metal,
with an acid.

6. A method for producing a compound represented by the formula (2), which comprises reacting a compound represented by the formula (2): 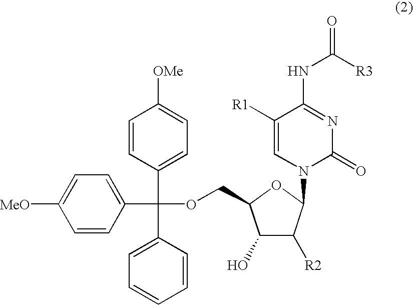
wherein R1 represents a hydrogen atom, an alkyl group having 1 to 4 carbon atoms, an alkenyl group having 2 to 4 carbon atoms, an alkynyl group having 2 to 4 carbon atoms, a perfluoroalkyl group having 1 to 4 carbon atoms, or a halogen atom; R2 is a hydrogen atom, an alkoxyl group having 1 to 4 carbon atoms, an alkoxyl group having 1 to 4 carbon atoms with a substituent, or a halogen atom; and R3 represents a methyl group or a phenyl group,
with a compound represented by the formula (3):
MX (3)
wherein M represents a positive ion of an alkali metal or an alkaline earth metal and X represents an alkoxide having 1 to 4 carbon atoms, an amide or a negative ion of a hydroxide ion,
or with a compound represented by the formula (4):
MY (4)
wherein M is as defined in M of the formula (3) and Y represents a halide ion,
in the presence of an organic amine followed by isolating a compound represented by the formula (1): 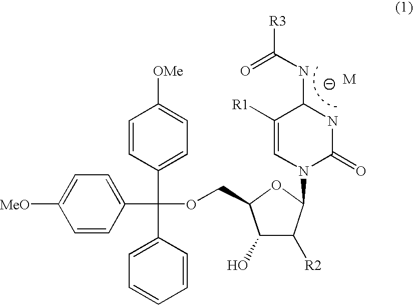
wherein R1, R2, and R3 are as defined in R1, R2, and R3 of the formula (2) and M is as defined in M of the formula (3),
and subsequently reacting the compound represented by the formula (1) with an acid to convert into the compound represented by the formula (2).

7. The method according to claim 4, wherein, in the formulae (1) and (2), R1 represents a hydrogen atom or a methyl group, R2 represents a hydrogen atom, and R3 represents a phenyl group.

8. The method according to claim 7, wherein, in the formula (1), M represents a lithium ion.

9. The compound according to claim 1, wherein, in the formula (1), M represents a lithium ion.

10. The method according to claim 5, wherein, in the formulae (1) and (2), R1 represents a hydrogen atom or a methyl group, R2 represents a hydrogen atom, and R3 represents a phenyl group.

11. The method according to claim 6, wherein, in the formulae (1) and (2), R1 represents a hydrogen atom or a methyl group, R2 represents a hydrogen atom, and R3 represents a phenyl group.

12. The method according to claim 4, wherein, in the formula (1), M represents a lithium ion.

13. The method according to claim 5, wherein, in the formula (1), M represents a lithium ion.

14. The method according to claim 6, wherein, in the formula (1), M represents a lithium ion.

15. The method according to claim 10, wherein, in the formula (1), M represents a lithium ion.

16. The method according to claim 11, wherein, in the formula (1), M represents a lithium ion.
